(19) United States
(12) Patent Application Publication
(10) Pub. No.: US 2007/0224160 A1
(43) Pub. Date: Sep. 27, 2007

(54) Nonleachable Composition and Method to Use

(75) Inventors: Tor P. Schultz, Starkville, MS (US); Darrel Nicholas, Starkville, MS (US); Stephen Scott Kelley, Cary, NC (US)

Correspondence Address: BUTLER, SNOW, O'MARA, STEVENS & CANNADA PLLC, 6075 POPLAR AVENUE, SUITE 500, MEMPHIS, TN, 38119, US

(73) Assignees: Mississippi State University, Mississippi State, MS (US); U.S. Department of Energy's National Renewable Energy Lab, Washington, DC (US)

(21) Appl. No.: 11/689,278

(22) Filed: Mar. 21, 2007

Related U.S. Application Data
(60) Provisional application No. 60/743,669, filed on Mar. 22, 2006.

(51) Int. Cl. A61K 31/74

(52) U.S. Cl. 424/78.08

(57) ABSTRACT

This invention relates to a nonleachable waterborne wood preservative composition that combines one or more resin acids, mostly or fully in the carboxylate anion form, with one or more organic biocides to enhance the efficacy of relatively high-cost organic biocides. Resin acids are cost effective and environmentally safe, and therefore can be used as a low-cost and environmentally-benign additive to help protect wood against wood-destroying fungi. Furthermore, wood treated with a waterborne resin acid formulation will have enhanced water repellency, which will result in better dimensional stability for wood exposed to water. This invention also provides a method for the use of the described composition.

### CROSS REFERENCE TO RELATED APPLICATIONS

This application claims the benefit of U.S. Ser. No. 60/743,669 filed Mar. 22, 2006, under 35 U.S.C. Section 1.119(e).

### FIELD OF THE INVENTION

This invention relates to a nonleachable waterborne composition of resin acids and wood preserving organic biocides and a method of preserving wood by contacting the nonleachable waterborne composition with wood.

### BACKGROUND OF THE INVENTION

Wood and wood derived products such as lumber, plywood, and laminated veneer are used for many applications such as home construction, fences, decks, poles, and railroad ties. Those wood products that are often wetted or maintain contact with soil are attacked and degraded by various organisms, including wood decaying fungi and insects. Wood biodeterioration causes extensive and costly damage to the associated wood structures, and therefore wood products are commonly treated with various organic biocides for protection against the aforementioned organisms. Many of the wood preservatives currently used to treat wood, however, have some environmental, disposal and health concerns associated with their use. For example, the wood preservative systems for residential exterior applications currently used in the U.S. are waterborne formulations based on copper(II) combined with a co-organic biocide to control copper tolerant fungi, with the biocides dissolved in a water carrier to give a liquid solution. The lumber or other wood product to be treated is often then put into a pressure treating cylinder where a vacuum is first drawn and then the biocide solution is pumped into the lumber-filled cylinder and the preservative liquid solution is pressurized which forces the preservative solution to penetrate into the lumber, known as the pressure treatment process. Alternatively, wood can be treated by dipping, spraying, or brushing the biocide solution onto the wood. Due to the low cost, safety, and absence of odors and oily residues, lumber for exterior residential applications are usually treated with waterborne formulations. Preservative solutions for industrial applications can be treated with an oilborne formulation, but the treated wood will have an unpleasant chemical odor and oily residue, and the oil solvent is more expensive than water.

However, lumber treated with the cooper-rich waterborne residential systems are known to leach relatively high levels of copper, therefore raising concerns regarding a possible negative impact on aquatic ecosystems, and disposal of metallic treated lumber is also an environmental concern. Based on these and other concerns, several European countries are mandating the use of totally-organic wood preservatives in residential applications. A few municipalities in the United States have also recently restricted the use of copper-treated wood and additional localities will likely enact restrictions in the near future. Although the total organic biocide systems currently being considered for use in the United States for exterior residential applications are effective wood preservatives, they are also relatively expensive compared to the current copper-based preservatives. Therefore, a need exists to develop environmentally-benign, effective, and economical organic waterborne wood preservative systems for exterior residential applications.

Wood is also a hydroscopic material so that in outdoor exposure wood products such as decking will sorb water during a rainstorm and swell, and then later when the sun dries out the wood the decking will shrink. Since wood is an anisotropic material which swells differently depending on the grain orientation, this leads to the wood decking having poor dimensional stability and so the decking will split, warp, cup, bow, etc.

### BRIEF SUMMARY OF THE INVENTION

This invention relates to an organic wood preservative that is a nonleachable waterborne composition made of an effective amount of at least one resin acid and an effective amount of at least one organic biocide in a waterborne formulation. Nonleachable means the at least one organic biocide and the salt of the at least one resin acid that are dissolved in water do not leach out of the waterborn wood preservative when applied as a wood preservative. More specifically, this invention related to a method of preserving wood involving the steps of contacting wood with an effective amount of at least one organic biocide and an effective amount of at least one resin acid in a waterborne system. In this method the wood can be preserved by the pressure treatment, immersion, dipping, spraying and brushing processes. In one embodiment the invention an effective amount of at least one resin acid in a waterborne formulation and an effective amount of at least one organic biocide in a waterborne or oilborne solvent are sequentially employed to treat the wood product known as a dual treatment, and in another embodiment the resin acid and organic biocide(s) are both combined together in a waterborne formulation which is employed simultaneously to treat the wood product by a pressure treating process, or dipping, spraying or brushing on the waterborne wood preservative.

### BRIEF DESCRIPTION OF SEVERAL VIEWS OF THE DRAWINGS

A more complete appreciation of the present invention and many attendant advantages thereof will be readily understood by reference to the following detailed description of the invention when considered in connection with the accompanying drawings, wherein:

FIG. 1 is a graph showing average compression strength of cottonwood wafers treated with the present composition of a resin acid and an organic biocide combined, and samples that were treated with only the organic biocide with the wood wafers treated by the two preservative systems then exposed to white rot decay fungus in the ager block laboratory decay test.

FIG. 2 is a graph showing average compression strength of cottonwood wafers treated with the present composition of a resin acid and an organic biocide combined, or treated with the biocide alone, with the wood wafers treated by the two preservatives exposed to white rot decay fungus in the ager block laboratory decay test.

FIG. 3 is a graph showing average compression strength of cottonwood wafers treated with an organic biocide alone, with half of the wafers then treated again with a waterborne resin acid solution, with the wood wafers then exposed to white rot decay fungus in the ager block laboratory decay test.

### DETAILED DESCRIPTION OF THE INVENTION

This invention relates to a nonleachable waterborne composition made of an effective amount of at least one resin acid, and an effective amount of at least one organic biocides to provide increased efficacy in protecting wood against degradation by wood destroying fungi and/or wood destroying insects such as termites.

The at least one organic biocide includes:
i. compounds from the class of biocides known as azoles, such as cyproconazole, propiconazole, and tebuconazole;
ii. 2-(thiocyanomethylthio)benzothiazole;
iii. 3-iodo-2-propynylbutyl carbamate;
iv. compounds from the class of organic biocide mixtures known as quaternary ammonium compounds;
v. chlorothalonil;
vi. dichlofluanid;
vii. 4,5-dichloro-2-n-octyl-4-isothiazoline-3-one;
viii. methylene bis-thiocyanate; and
ix. or other organic biocides known to be effective at protecting wood against fungal and/or insect degradation.

An effective amount of the at least one organic biocide is between 0.001% to 10% by weight of the nonleachable composition that is applied to the wood.

Resin acids are a chief component of gum, wood, and tall oil rosin. Resin acids are natural terpenoid compounds that are produced by parenchymatous epithelial cells that surround the resin ducts in coniferous trees such as pines. The terpenoids, which includes resin acids and other compounds such as monoterpenes, are formed when isoprene building units couple to form mono-, sesqui-, and diterpene structures. Resin acids have two functional groups, carboxyl group and double bonds. Nearly all have the same basic skeleton: a 3-ring fused system with the empirical formula C₁₉H₂₉COOH. Resin acids are part of the family of organic acids remaining once the terpene components such as monoterpenes and other non-terpenoid extractives such as the fats and fatty acids have been removed from solidified resin commonly obtained from plants, trees, pine stumps, guayule plants, pine bark, or as a byproduct of chemically pulped wood. Examples of resin acids include the abietanes, such as abietic acid and neoabietic acid as well as the tricyclic diterpenoids, such as, pimaric acid. The term resin acids also includes resin acid derivatives. Resin acids can be chemically modified to rosin esters and rosin maleics are examples of resin acid derivatives. The purity of the at least one resin acid is between 40% and 100%.

The concentration of the at least one resin acid is between 0.1 to 25% by weight of the nonleachable composition. The nonleachable composition may further include an effective amount of a nonbiocidal additive, such as, an antioxidant, dye, antifoaming agent or other nonbiocidal additives known to provide beneficial benefits to treated wood.

This invention claims that a non-leachable waterborne wood preservative formulation containing a combination of resin acids, mainly or fully in the carboxylate anion form, and one or more organic biocides increases the efficacy of the organic biocide(s) in protecting the wood against biodegradation. In addition, the treated wood maintains a degree of water repellency that improves the dimensional stability of wood exposed to water.

An effective amount of the at least one organic biocide is between 0.001% to 10% by weight of the nonleachable composition that is applied to the wood.

The at least one organic biocide and a salt of at least one resin acid are solubilized in a liquid carrier medium. The liquid carrier is mostly water but can include an organic co-solvent that is soluble in water, such as isopropanol. The function of the organic co-solvent is to assist in solubilizing the resin acid salt.

Preparation of the organic wood preservative waterborne composition is based upon the formation of the water soluble salt of the resin acid. In the preferred embodiment the salt is the sodium form. This salt form of the resin acid acts a surfactant, and thereby allows the incorporation of water-insoluble organic biocides into the waterborne composition. Once the waterborne resin acid salt formulation is impregnated into wood via a pressure treatment or other processes such as dipping, brushing, or spraying the naturally acidic wood converts the carboxylate anion back to the acid form so that the added resin acid does not leach from the treated wood. In addition, depositing the hydrophobic resin acid into wood, results in the wood having enhanced water repellency and, thus, greater dimensional stability in outdoor exposure. Alternatively, wood can be separately treated with the waterborne resin acid carboxylate anion solution and a separate solution of a waterborne or solvent borne organic biocide, known as a dual treatment. Compositions of this invention may be applied to the wood to be treated using a variety of well known processes such as dipping, spraying, brushing, pressure treatment and the like.

### EXAMPLE 1

A 4% abietic acid solution, the most common resin acid in Tall Oil Rosin (TOR), was made as follows. Forty grams of technical grade abietic acid was ground to a powder, and added to a flask. Then, 150 mL of isopropanol, and 400 mL of water were added. Finally, 125 mL of 1.0 N NaOH solution followed by 435 mL of more water. The solution was stirred overnight with a magnetic stirrer to give a brownish solution with a pH of about 8.

Two sticks of defect-free cottonwood wapwood were cut into wafers five mm thick (longitudinal dimension) by 18 mm×18 mm. The wafers were sequentially numbered as they were cut, then randomized, and finally divided into sets. Each set had eight wafers. Wafers that were all cut from one stick were used throughout the experiment for one biocide, with the wafers from the other stick used for the other biocide. Each set of eight wafers was treated by a full cell process, with a particular biocide level in the treatment solution expressed in percent. The samples were weighed before and after the full cell treatment, and the biocide retention (pcf) was calculated. Treatment of the cottonwood wafers by a full cell process with only the 4% resin acid treatment resulted in an abietic acid retention of approximately 1.8 pounds per cubic foot (pcf). Two biocides were separately examined, the isothiazolone 4,5-dichloro-2-n-octyl-4-isothiazolin-3-one, also called RH-287, and the azole propiconazole. The particular biocide was dissolved in the 4% waterborne resin acid solution acid solution for the wafers treated with both the biocide and resin acid, or dissolved into a 40:60 isopropanol:water solution for the samples treated with only the biocide. The waters were treated with solutions of three different biocide concentrations, each with or without co-added resin acid. After treatment the wafers were allowed to dry, then sterilized and put into an agar block laboratory decay test using the decay fungus Trametes versicolor. The samples were incubated for six weeks.

Upon completion of the incubation period the samples were removed and the compression strength of the water-saturated wafers was determined, where a decrease in strength indicated an increase in fungal degredation.

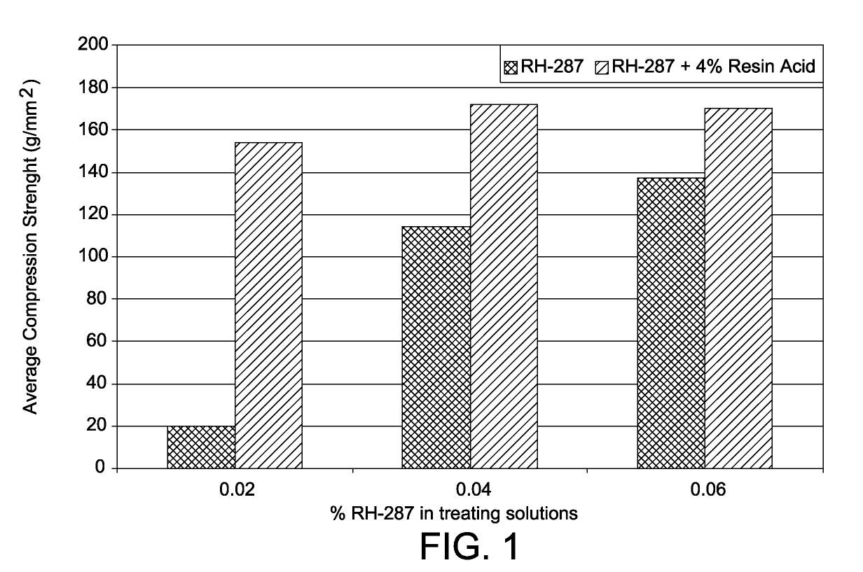

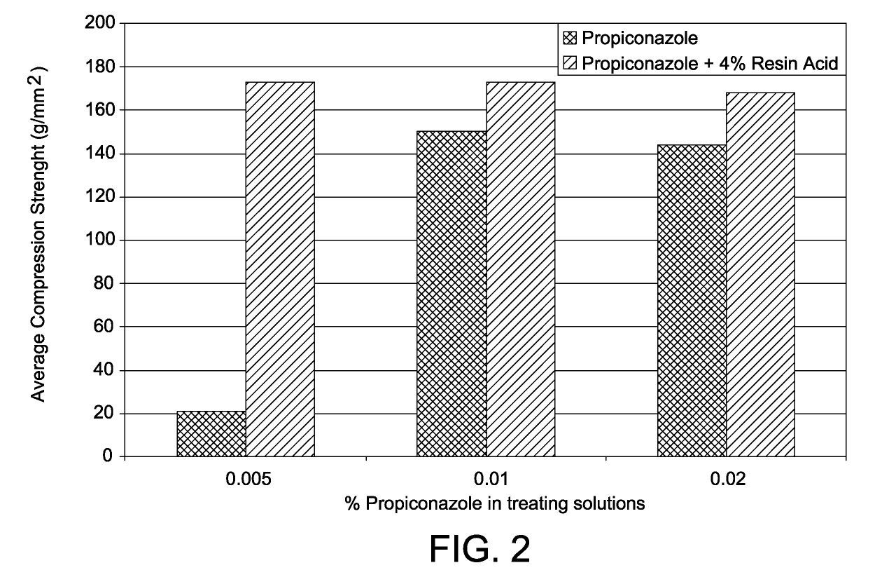

Now referring to FIGS. 1-2, the average strength of the samples treated with the two biocides and 4% resin acid was always greater than the strength of wafers treated with the same retention of the organic biocide alone. Thus, the combination of the organic biocide and carboxylate anion of the resin acid had greater efficacy in protecting the wood against fungal degradation than the organic biocide alone at the same treatment level for both biocides examined.

### EXAMPLE 2

A 5% abietic acid solution was prepared by initially grinding fifty grams of abietic acid to a powder, and then adding the powder to a flask. To the flask, 60 mL of isopropanol and 250 mL of water were added before adding 154 grams of 1.0 N NaOH solution. Finally, 486 mL of water was added to the flask and the solution was stirred overnight to give a clear brown solution with a pH of approximately eight.

Defect-free cottonwood sapwood wafers 5 mm thick (longitudinal dimension) by 18 mm×18 mm were treated by a full-cell process with 0.1, 0.2, and 0.4% of the organic biocide chlorothalonil (CTN) dissolved in toluene. The samples were then dried, and half of the treated wafers were re-treated with the waterborne 5% abietic acid solution described above. The sample, both those treated with the organic biocide alone and those dual treated with both the biocide followed by abietic acid, were then tested by the agar block laboratory decay test using the white-rot decay fungus Trametes versicolor and incubated for seven weeks.

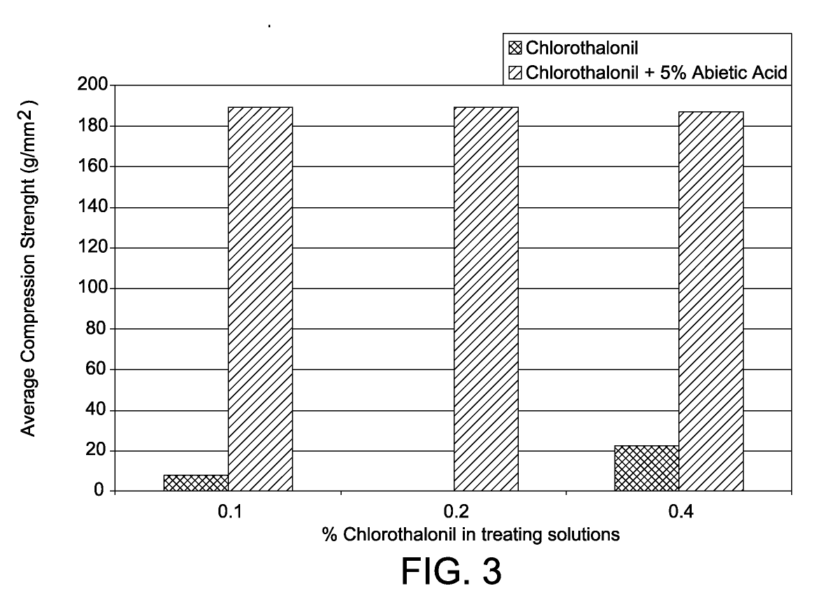

Upon completion of the incubation period, the wafers were removed from the incubator and the compression strength was determined, where a decrease in strength indicating an increase in the fungal degradation of the wood. For all three biocide concentrations examined the presence of 5% abietic acid plus the biocide resulted in significantly higher strength that the samples treated with only the organic biocide chlorothalonil at the same retention. Thus, we conclude that the co-addition of 5% abietic acid increased the efficacy of the organic biocide at protecting the wood against fungal degradation as shown in FIG. 3.

### EXAMPLE 3

A wax solution was prepared by dissolving 1.1 grams of paraffin wax into 100 mL of toluene. A 1.0% abietic acid waterborne solution was prepared by combing 1.0 grams of abietic acid powder, 6 mL of isopropanol, 90 mL deionized water, and 3.1 mL of 1.0N NaOH, with the mixture stirred overnight. A slightly higher concentration of wax was employed than resin acid to ensure that any benefit of the resin acid compared to the wax treatment would be definitive.

Southern yellow sapwood pine wafers (cut from defect-free sapwood), 5 mm×19 mm×40 mm [tangential×radical×longitudinal], were used for all treatment tests. A 1.1% wax/toluene solution was used to treat wafers by a full cell process and then air-dried at room temperature and then placed in a 12% equilibrium moisture content (EMC) room. Matched controls were untreated. In addition, a second set of the pine wafers was treated with the 1.0% abietic acid/water solution by a full cell process and then air-dried and sequentially placed in a 12% EMC room. The matching wafers were untreated, and placed in a 12% EMC room. Once conditioned to 12% EMC, sets of the wood samples (treated sample and matched control) were immersed in water side-by-side and the radial swell measured over a 90 minute period. The reduction in the radial swelling relative to the matched untreated control wafer is called the water repellency effiency, or WRE, and reported in percent. Percent WRE was determined as [(untreated radical swell−treated radical swell)/untreated radical swell]*100. A % WRE that is zero (or close to zero) represents a treatment that had no effect on reducing water-caused swelling. Alternatively, a % WRE greater than zero represents a positive effect on the dimensional stability of the wafers as a result of the treatment process.

The average % WRE at 10, 31, and 91 minutes for four sets of each treatment (1.1% wax/toluene; and 1.0% abietic acid/water, with a matched untreated control run side-by-side each time) is shown in Table 1. In summary, after the 10 minute immersion time, the two treated samples showed comparable properties of water repellency. However, at the longer water immersion time of 31 minutes, the 1.0% abietic acid was approximately twice as effective a water repellent as 1.1% wax. Finally, the longest water immersion time of 91 minutes resulted in a total loss of water repellency effectiveness with the wax treatment while the abietic acid treated samples maintained some degree of dimensional stability.

TABLE 1

| | Average % WRE | | |
|---|---|---|---|
| Formulation | 10 Min | 31 Min | 91 Min |
| 1.1% Paraffin | 49.2 | 21.9 | −20.2 |
| Wax/Toluene | | | |
| 1.0% Abietic | 50.0 | 40.2 | 22.0 |
| Acid/Water | | | |

### EXAMPLE 4

Solid tall oil rosin (TOR) (Pamite 90™; reportedly 92.7% resin acids, approximately 1.1% fatty acids, and various percentages of other neutral unidentified compounds) was initially ground to a fine powder. Three hundred and eighty eight grams of the powder was added to a 5 gallon container along with 5,394 grams of water, 720 grams of isopropanol, 1,200 grams of 1.0 N NaOH, 6 grams of the azole biocide tebaconazole, and 5,000 additional grams of water for preparation of the treatment solution. The mixture was initially stirred for approximately three hours (pH=9), and then overnight to eliminate any solid particles. The final pH of the solution was approximately eight.

Twelve southern yellow pine decking boards (defect free sapwood) 1.25 inches thick (nominal)×6 inches wide (nominal)×4 feet in length were cut to obtain two matching samples, each 22 inches long. One board from each matched set was treated with the above solution by a full cell process with the other matched sample untreated. The sets of treated and untreated decking boards were then placed on top of a black roof, slanted and facing to the south for maximum sunlight exposure for five weeks. The combined effect of weather/rain exposure and the high temperatures (150° F.) on the roof provided an ideal environment to test the water repellency of the wood, as wood in these severe exposure conditions are more prone to photodegradation and cracking/splitting.

After the five weeks of outdoor exposure period and following a summer rainstorm, the average % moisture content of the TOR-treated boards was found to be 25.3%, and the average % moisture content of the control boards was 31.0%, and the outdoor exposure test was continued. The lower retained water content of the TOR-treated boards illustrates the water repellent nature of the treated wood. This effect is obviously advantageous as an increase in water repellency would result in a lower decay potential, reduced biocide leaching, and increased dimensional stability (less splitting, warping, checking, etc.) in an above-ground exposure such as decking. For example, after 15 months of exposure the aboveground outdoor exposed boards were examined for checking using a 0 to 4 rating system, with a “0” rating given to samples with no splitting and a “4” rating for boards with such severe checking that the structural properties of the board was affected. The untreated boards had an average checking rating of 2.9, while the TOR-treated boards had a lower check rating of 1.6. Thus, less checking occurred with the TOR-treated boards, showing that the water repellency of the TOR treatment resulted in greater dimensional stability of boards exposed outdoors.

The above detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

1. A nonleachable composition consisting essentially of an effective amount of at least one organic biocide, a carboxylated anion solution of at least one resin acid and liquid carrier medium.

2. The composition of claim 1 wherein the at least one organic biocide is selected from the group consisting of:
i. an azole biocide;
ii. 2-(thiocyanomethylthio)benzothiazole;
iii. 3-iodo-2-propynylbutyl carbamate;
iv. a quaternary ammonium biocide;
v. chlorothalonil;
vi. dichlofluanid;
vii. 4,5-dichloro-2-n-octyl-4-isothiazoline-3-one; and
viii. methylene bis-thiocyanate.

3. The composition of claim 1, further comprising an effective amount of nonbiocidal additive.

4. The composition of claim 1, wherein the purity of the at least one resin acid is between 40% to 100%.

5. The composition of claim 1, further comprising a liquid carrier medium of at least 40% water and an organic co-solvent that is soluble in water.

6. The composition of claim 2, wherein the azole is selected from the group consisting of cyproconazole, propiconazole and tebuconazole.

7. The composition of claim 1, wherein the concentration of the at least one organic biocide is between 0.0001% to 10% by weight of the nonleachable composition that will be employed to treat the wood.

8. The composition of claim 1, wherein the concentration of the at least one resin acid is between 0.1 to 25% by weight of the nonleachable composition.

9. A method of preventing degradation of wood product by comprising: treating a wood product with effective amount of the nonleachable composition of claim 1.

10. The method of claim 9 wherein the at least one organic biocide is selected from the group consisting of:
ix. an azole biocide;
x. 2-(thiocyanomethylthio)benzothiazole;
xi. 3-iodo-2-propynylbutyl carbamate;
xii. a quaternary ammonium biocide;
xiii. chlorothalonil;
xiv. dichlofluanid;
xv. 4,5-dichloro-2-n-octyl-4-isothiazoline-3-one; and
xvi. methylene bis-thiocyanate.

11. A method to prevent the degradation of wood product comprising the steps of:
a) treating a wood product with an aqueous solution comprising at least one resin acid carboxylate anion for a sufficient amount of time for said aqueous solution to be adsorbed by said wood product; and
b) treating said wood product with at least one organic biocide in solution for a sufficient amount of time for said solution to be adsorbed by said wood product.

12. The method of claim 11 wherein the at least one organic biocide is selected from the group consisting of:
xvii. an azole biocide;
xviii. 2-(thiocyanomethylthio)benzothiazole;
xix. 3-iodo-2-propynylbutyl carbamate;
xx. a quaternary ammonium biocide;
xxi. chlorothalonil;
xxii. dichlofluanid;
xxiii. 4,5-dichloro-2-n-octyl-4-isothiazoline-3-one; and
xxiv. methylene bis-thiocyanate.

13. A method to make a nonleachable composition comprising the steps of:
admixing at least one organic biocide, the salt of at least one resin acid and a liquid carrier medium to form a nonleachable composition.

14. The method of claim 13 wherein the at least one organic biocide is selected from the group consisting of:
i. an azole biocide;
ii. 2-(thiocyanomethylthio)benzothiazole;
iii. 3-iodo-2-propynylbutyl carbamate;
iv. a quaternary ammonium biocide;
v. chlorothalonil;
vi. dichlofluanid;
vii. 4,5-dichloro-2-n-octyl-4-isothiazoline-3-one; and
viii. methylene bis-thiocyanate.
